(19) United States
(12) Patent Application Publication
(10) Pub. No.: US 2003/0068318 A1
(43) Pub. Date: Apr. 10, 2003

(54) Treatment of uterine serous papillary cancer

(76) Inventors: Timothy O'Brien, Little Rock, AR (US); Stefania Bellone, Little Rock, AR (US); Martin J. Cannon, Little Rock, AR (US); Alessandro D. Santin, Little Rock, AR (US)

Correspondence Address: Benjamin Aaron Adler, ADLER & ASSOCIATES, 8011 Candle Lane, Houston, TX, 77071, US

(21) Appl. No.: 09/967,263

(22) Filed: Sep. 28, 2001

(51) Int. Cl. A61K 39/395

(52) U.S. Cl. 424/141.1; 424/155.1

(57) ABSTRACT

The present invention provides method of treating uterine serous papillary carcinoma in an individual in need of such treatment, comprising the step of administering to said individual a therapeutically effective dose of a HER-2/neu antibody. Also provided is a method of differentiating primary uterine serous papillary carcinoma from serous papillary ovarian tumors in an individual, comprising the step of measuring the expression of HER-2/neu in said tissue, wherein the presence of an increased and constitutive expression pattern in said tissue indicates that said tumor is a uterine serous papillary carcinoma. Further provided is a method of treating uterine serous papillary carcinoma in an individual in need of such treatment, comprising the step of administering to said individual a therapeutically effective dose of a HER-2/neu antibody and a therapeutically effective dose of interleukin-2.

### BACKGROUND OF THE INVENTION

1. Field of the Invention

The present invention relates generally to the field of gynecological oncology. More specifically, the present invention relates to the treatment of uterine serous papillary cancer.

2. Description of the Related Art

Cancer of the uterine corpus represents the most common gynecologic malignancy with approximately 37,400 new cases diagnosed in the United States in 1999 (1). Uterine serous papillary carcinoma (USPC)² is a histologic subtype of endometrial cancer constituting up to 10% of all endometrial cancers. Histologically similar to high grade ovarian cancer (2,3), uterine serous papillary carcinoma has a propensity for early intra-abdominal and lymphatic spread even at presentation (4,5) and is characterized by a highly aggressive biologic behavior (1-4). In contrast to ovarian cancer, however, it is a chemoresistant disease since its onset with responses to combined cisplatinum-based chemotherapy in the order of 20% and of short duration (6,7).

The survival rate for uterine serous papillary carcinoma is dismal, even when uterine serous papillary carcinoma is only a minor component of the histologically more common endometrioid adenocarcinoma (3,5). The overall 5-year survival is 30%±9% for all stages and the recurrence rate after surgery is extremely high (50% to 80%). Novel therapeutic strategies effective in the treatment of residual and/or metastatic uterine serous papillary carcinoma are desperately needed.

Proto-oncogenes are a group of normal genes that play important roles in the regulation of cell proliferation. Abnormalities in the expression, structure, or activity of proto-oncogene products contribute to the development and maintenance of the malignant phenotype. The human HER-2/neu (c-erbB2) gene product, like the epidermal growth factor receptor, is a transmembrane receptor protein that includes a cysteine rich extracellular ligand binding domain, a hydrophobic membrane spanning region, and an intracellular tyrosine kinase domain (8). With no direct ligand identified to date, HER-2/neu functions as a preferred partner for heterodimerization with other members of the EGFR family (namely HER-1 or ErbB1, HER-3 or ErbB3 and HER-4 or ErbB4), and thus plays an important role in coordinating the complex ErbB signaling network that is responsible for regulating cell growth and differentiation (9-10).

Initially identified as the proto-oncogene associated with the development of neuroblastomas in rats exposed to ethylnitrosurea in utero (11), HER-2/neu has subsequently been shown to be overexpressed in approximately one-third of primary ovarian carcinomas and breast carcinomas as well as other human tumors including colon, lung, prostate and cervical cancers (12). In breast and ovarian cancer, several, but not all studies have reported that the amplification of this gene is associated with resistance to treatment and poor survival, suggesting that cells overexpressing HER-2/neu may manifest a more aggressive biologic behavior and may have a selective growth advantage over HER-2/neu-negative tumor cells (11, 13-16).

Recently, a humanized monoclonal antibody (MAb) to HER-2/neu, Herceptin, has been reported to have significant therapeutic effects in patients with strongly (i.e., score 2+ and 3+) HER-2/neu-positive breast carcinomas, particularly when combined with chemotherapeutic drugs (17,18). In ovarian, lung cancer and prostate cancers, clinical studies are currently investigating the efficacy of Herceptin in patients whose tumors exhibit strong plasmalemmal immunoreactivity for this protein (19). In contrast to ovarian cancer, however, very little is known about HER-2/neu expression by the histologically similar but biologically more aggressive uterine serous papillary carcinoma.

The prior art is deficient in the lack of an effective treatment for uterine serous papillary carcinoma. The present invention fulfills this long-standing need and desire in the art.

### SUMMARY OF THE INVENTION

The present invention reports that a variant of uterine cancer commonly overexpresses HER-2/neu (i.e., score 2+ or more in 80% of samples tested), and that the levels of protein expression on primary uterine serous papillary carcinoma cell lines recorded by flow cytometry are on average 10-fold higher when compared to fresh or established breast and ovarian HER-2/neu positive cancer cell lines. Importantly, although uterine serous papillary carcinoma cell lines are resistant to natural killer dependent cytotoxicity in vitro, they retain high sensitivity to anti-HER-2/neu antibody dependent cellular cytotoxicity (ADCC), and that their in vitro proliferation is significantly inhibited by anti-HER2/neu monoclonal antibody (Herceptin®). Furthermore, a significant enhancement of antibody dependent cellular cytotoxicity was demonstrated when peripheral blood effector cells were incubated with uterine serous papillary carcinoma cells in the presence of low doses of IL-2. Thus, Herceptin® therapy is a therapeutic strategy in patients harboring this biologically aggressive and chemotherapy and radiotherapy-resistant variant of endometrial cancer.

In one embodiment of the instant invention, a method of treating uterine serous papillary carcinoma in an individual in need of such treatment, comprising the step of administering to said individual a therapeutically effective dose of a HER-2/neu antibody.

In another embodiment of the instant invention, a method is provided of differentiating primary uterine serous papillary carcinoma from serous papillary ovarian tumors in an individual, comprising the step of measuring the expression of HER-2/neu in said tissue, wherein the presence of an increased and constitutive expression pattern in said tissue indicates that said tumor is a uterine serous papillary carcinoma.

In yet another embodiment of the instant invention, a method of treating uterine serous papillary carcinoma in an individual in need of such treatment, comprising the step of administering to said individual a therapeutically effective dose of a HER-2/neu antibody and a therapeutically effective dose of interleukin-2.

Moreover, further aspects will be apparent from the following description of the embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate embodiments of the invention and therefore are not to be considered limiting in their scope.

FIG. 1 shows representative hematoxylin and eosin sections of USPC-4 and USPC-1 which stained light (1+) for HER-2/neu (FIG. 1A) and heavy (3+) for HER-2/neu (FIG. 1B), respectively. FIG. 1C, FIG. 1D, FIG. 1E, FIG. 1F: Immunohistochemical staining for HER-2/neu expression on paraffin embedded uterine serous papillary carcinoma specimens. (FIG. 1C) USPC-4 with light (1+) staining for HER2/neu. (FIG. 1D, FIG. 1E, FIG. 1F) USPC-1, USPC-2, and USPC-3, respectively, from which heavy (3+) staining for HER-2/neu was detected. Original magnification 400×.

FIG. 2 shows a representative FACS analysis of Herceptin® staining of primary uterine serous papillary carcinoma cells, primary and established ovarian and breast cancer cell lines. Data with Herceptin® are shown in solid black while isotype control monoclonal antibody profile are shown in white. Similar results were obtained with FITC-labeled antiHER-2/neu monoclonal antibody (Oncogene Science) stained tumor cell lines (data not shown). HER-2/neu expression was significantly higher on uterine serous papillary carcinoma cell lines compared to fresh and established ovarian cancer cell lines and established breast cancer cell lines (p<0.001 by student t test)

FIG. 3 shows antibody dependent cellular cytotoxicity mediated by Herceptin® (2 mg/ml) against ⁵Cr-labeled USPC-1 (upper panel) and USPC-2 (lower panel) cells (10,000 cells/sample), as measured in combination with effector peripheral blood lymphocytes from a representative healthy, heterologous donor in a 5 hrs assay. The figure shows the percentage of target cell lysis (±SD). Effector cells plus Rituxan® (2 mg/ml) were used as controls. Similar antibody dependent cellular cytotoxicity results were obtained with the use of Herceptin® at 1 or 5 mg/ml (data not shown).

FIG. 4 shows antibody dependent cellular cytotoxicity mediated by Herceptin® (2 mg/ml) against ⁵¹Cr-labeled USPC-3 cells (10,000 cells/sample), as measured in combination with autologous effector peripheral blood lymphocytes in a 5 hr assay. The figure shows the percentage of target cell lysis (±SD). Peripheral blood lymphocytes plus medium only, or Rituxan® (2 mg/ml), were used as controls. Similar antibody dependent cellular cytotoxicity results were obtained with the use of Herceptin® at 5 mg/ml (data not shown).

FIG. 5 shows the effect of complement and serum immunoglobulin (dilution 1:2) on cytotoxicity mediated by Herceptin® (2 mg/ml) against ⁵¹Cr-labeled USPC-1 (FIG. 5A, FIG. 5C, FIG. 5E) and USPC-2 (FIG. 5B, FIG. 5D, FIG. 5F) cells (10,000 cells/sample), measured in the presence or absence of effector peripheral blood lymphocytes from a representative heterologous healthy donor in a 5 hr assay. The figure shows the percentage of target cell lysis (±SD) at E/T ratios 25:1. Effector peripheral blood lymphocytes with medium alone, or with Rituxan® (2 mg/ml) plus or minus serum were used as controls. Herceptin®-mediated antibody dependent cellular cytotoxicity in the presence of heat-inactivated human serum and effector peripheral blood lymphocytes was not significantly different to the results obtained in the absence of serum. Herceptin®-mediated antibody dependent cellular cytotoxicity in the presence of untreated human serum and effector peripheral blood lymphocytes was significantly increased compared to the results obtained in the absence of serum (p<0.03).

FIG. 6 shows enhancement of antibody dependent cellular cytotoxicity mediated by Herceptin® (2 mg/ml) against ⁵¹Cr-labeled USPC-2 cells (10,000 cells/sample) in the presence of 100 IU/ml of IL-2 for 5 hr (upper panel) or following pre-incubation of effector peripheral blood lymphocytes with 100 IU/ml of IL-2 for 72 hr (lower panel), as measured in a 5 hr assay. The figure shows the percentage of target cells lysis (±SD) at E/T ratios 50:1. Effector peripheral blood lymphocytes with medium only, or in the presence of Rituxan® (2 mg/ml) were used as controls. Herceptin-mediated antibody dependent cellular cytotoxicity was significantly enhanced (p<0.01 by student t test). A small but significant increase in cytotoxic activity was seen at 72 hr of IL-2 exposure in the absence of Herceptin® and in the presence of Rituxan® (p<0.05). Similar results were obtained after incubation of effector peripheral blood lymphocytes with 50 IU/ml of IL-2.

### DETAILED DESCRIPTION OF THE INVENTION

The instant invention is directed to a method of treating uterine serous papillary carcinoma in an individual in need of such treatment, comprising the step of administering to said individual a therapeutically effective dose of a HER-2/neu antibody. Preferably, the antibody is a monoclonal antibody and even more preferably the antibody is a humanized monoclonal antibody. A representative example of an antibody useful in the methods of the present invention is Herceptin®. A person having ordinary skill in this art would readily recognize suitable dosages of an antibody useful in these methods. For example, the Herceptin® antibody is administered in a dose of from about 4 mg/kg to about 8 mg/kg. This method further comprises the step of administering a therapeutically effective dose of interleukin-2 to said individual. Generally, the interleukin-2 is recombinant interleukin-2. Preferably, the dose of interleukin-2 is very low and non-toxic. For example, interleukin-2 may be administered to an individual in a dose of from about 1×10⁶ IU/M² to about 10×10⁶ IU/M².

The instant invention is also directed to a method of differentiating primary uterine serous papillary carcinoma from serous papillary ovarian tumors in an individual, comprising the step of measuring the expression of HER-2/neu in said tissue, wherein the presence of an increased and constitutive expression pattern in said tissue indicates that said tumor is a uterine serous papillary carcinoma.

The instant invention is also directed to a method of treating uterine serous papillary carcinoma in an individual in need of such treatment, comprising the step of administering to said individual a therapeutically effective dose of a HER-2/neu antibody and a therapeutically effective dose of interleukin-2. Preferably, the HER-2/neu antibody is a monoclonal antibody and even more preferably is a humanized monoclonal antibody. Most preferably, the HER-2/neu antibody is Herceptin®. Generally, the HER-2/neu antibody may be given in any therapeutically effective dose but preferably the antibody is administered to the individual in a dose of from about from about 4 mg/kg to about 8 mg/kg. Preferably, the interleukin-2 is recombinant interleukin-2 and is administered to the individual in a dose that is non-toxic, for exampl, a dose of from 1×10⁶ IU/M² to about 10×10⁶ IU/M².

The following examples are given for the purpose of illustrating various embodiments of the invention and are not meant to limit the present invention in any fashion.

### EXAMPLE 1

Immunostaining of Formalin-fixed Tumor Tissues

Formalin-fixed, paraffin-embedded tissue blocks from 10 uterine serous papillary carcinoma cases were retrieved from the surgical pathology files of the University of Arkansas for Medical Sciences (UAMS), Little Rock, Ark. Study blocks were selected after histopathologic review by a surgical pathologist from patients who underwent primary surgical therapy for invasive uterine serous papillary carcinoma at UAMS between 1998 to 2001. Tumors were staged according to the F.I.G.O. operative staging system. Patient characteristics are described in Table 1.

TABLE 1
Base-line characteristics of the patients

| | | | | | Her2/neu |
|---|---|---|---|---|---|
| Patients | Age | Race | Year Diagnosed | Stage | positivity |
| USPC-1 | 62 | A-A | 1998 | IVa | 3+ |
| USPC-2 | 63 | A-A | 1998 | IVb | 3+ |
| USPC-3 | 59 | C | 2001 | IVb | 3+ |
| USPC-4 | 73 | C | 2000 | Ib | 1+ |
| USPC-5 | 73 | C | 1999 | IIb | 3+ |
| USPC-6 | 62 | A-A | 2000 | Ia | 3+ |
| USPC-7 | 58 | A-A | 1998 | Ib | 3+ |
| USPC-8 | 63 | A-A | 2000 | IIIc | 3+ |
| USPC-9 | 63 | C | 1999 | IIIa | 3+ |
| USPC-10 | 64 | A-A | 2000 | IVa | 3+ |

C: caucasian;
A-A: Afro-American

Total abdominal hysterectomy and regional lymph node sampling for invasive uterine serous papillary carcinoma were performed in all cases. In addition, in an attempt to established primary uterine serous papillary carcinoma tumor cell lines from these patients, 4 uterine serous papillary carcinoma fresh tumor biopsies were obtained at the time of surgery through the Gynecologic Oncology Division and the Pathology Department of UAMS, under approval of the Institutional Review Board.

The level of expression of HER-2/neu was evaluated b y standard immunohistochemical staining by an external independent laboratory (PhenoPath Laboratories, Seattle Wash.). The most representative hematoxylin and eosin-stained block sections were used for each specimen. When available, both primary and metastatic sites were evaluated for HER-2/neu expression. Briefly, immunohistochemical stains were performed on 4 mm-thick sections of formalin-fixed, paraffin embedded tissue. After pretreatment with 10 mM citrate buffer at pH 6.0 using a steamer, they were incubated with anti-HER-2/neu monoclonal antibody (DAKO, Glostrup, Denmark) at 1:2000 dilution. Slides were subsequentally labelled with streptavidin-biotin (LSAB)(DAKO, Glostrup, Denmark), stained with diaminobenzidine and counterstained with hematoxylin. The intensity of staining was graded as 0 (staining not greater than negative control), 1+(light staining), 2+(moderate staining), or 3+(heavy staining).

### EXAMPLE 2

Establishment of Uterine Serous Papillary Carcinoma Cell Lines

Three primary uterine serous papillary carcinoma cell lines (USPC-1, USPC-2 and USPC-3) were established after sterile processing of the tumor samples from surgical biopsies as previously described for ovarian carcinoma specimens (20). Primary uterine serous papillary carcinoma cell lines were analyzed by flow cytometry for HER-2/neu expression immediately after tumor processing, and after in vitro culture from 1 week to over 3 years (USPC-1 and USPC-2). Similarly, 2 primary serous papillary ovarian carcinoma cell lines established in the laboratory during the same study period from advanced stage ovarian cancer patients (OVA-4 and OVA-5) as well as 2 established and previously characterized serous ovarian cancer (UCI-101 and UCI-107, provided by Dr. Alberto Manetta, University of California, Irvine) and breast cancer cell lines (B7-474 and SK-BR-3; American Type Culture Collection, A.T.C.C.), the latter previously shown to highly over-express HER2/neu (21), were analyzed as positive controls by flow cytometry for HER-2/neu expression (see below).

### EXAMPLE 3

Flow Cytometry

The clinically marketed anti-HER-2/neu monoclonal antibody Herceptin® (Genentech, San Francisco, Calif.) was used for most of the study. For comparison, an unconjugated anti-HER2/neu (mouse IgG1) monoclonal antibody obtained from Oncogene Science (Uniondale, N.Y.) was used. Herceptin® is an IgG1k that contains human framework regions with the complementary-determining regions of a murine monoclonal antibody that binds to the Mr 185,000 extracellular determinant of HER-2/neu. For staining by Herceptin®, a FITC-conjugated goat anti-human F(ab¹)2 immunoglobulin was used as a secondary reagent (BioSource International, Camarillo, Calif.). For staining by unconjugated anti-HER-2/neu (mouse IgG1) a goat anti-murine FITC labeled mouse IgG1 (Beckman-Coulter Miami, Fla.) was used. Analysis was conducted with a FACScan, utilizing cell Quest software (Beckton Dickinson).

### EXAMPLE 4

Tests for Antibody Dependent Cellular Cytotoxicity

A standard 5-hour chromium (⁵¹Cr) release assay was performed to measure the cytotoxic reactivity of Ficoll-Hypaque separated peripheral blood lymphocytes (PBL) from several healthy donors and one uterine serous papillary carcinoma patient in combination with Herceptin against tumor target cell lines. The release of ⁵¹Cr from the target cells was measured as described (22) 1 0 as evidence of tumor cell lysis, after exposure of tumor cells to varying concentrations of Herceptin (ranging from 1 mg/ml to 5 mg/ml). Controls included the incubation of target cells alone or with peripheral blood lymphocytes or monoclonal antibody separately. The chimeric anti-CD20 monoclonal antibody Rituximab (Rituxan, Genentech, Calif.) was used as control for Herceptin in all bioassays. antibody dependent cellular cytotoxicity was calculated as the percentage of killing of target cells observed with monoclonal antibody plus effector cells, as compared with ⁵¹Cr release from target cells incubated alone.

### EXAMPLE 5

Test for Complement-Mediated Target-Cell Lysis and G-globulin Inhibition

A standard 5-hour chromium (⁵¹Cr) release assay identical to those used for antibody dependent cellular cytotoxicity assays, except that human serum (as a source of complement) diluted 1:2 to 1:4 was added in place of the effector cells, was used to test for complement-mediated target cell lysis. To test for the possible inhibition of antibody dependent cellular cytotoxicity against uterine serous papillary carcinoma cell lines by physiological human plasma concentrations of γ-globulin, heat inactivated (56° C. for 30 min) human serum was diluted 1:2 to 1:4 before being added in the presence or absence of effector peripheral blood lymphocytes. In some experiments, non-heat-inactivated human serum (diluted 1:2 or 1:4) was added in the presence of effector peripheral blood lymphocytes. Controls included the incubation of target cells alone or with either lymphocytes or monoclonal antibody separately. Rituxan was used as control MAb.

### EXAMPLE 6

IL-2 Enhancement of Antibody Dependent Cellular Cytotoxicity

To investigate the effect of IL-2 on Herceptin-mediated antibody dependent cellular cytotoxicity, effector peripheral blood lymphocytes were incubated at 37° C. at a final concentration of IL-2 (Aldesleukin, Chiron Therapeutics, Emeryville, Calif.) ranging from 50 to 100 IU/ml in 96-well microtiter plates. In some experiments, effector peripheral blood lymphocytes were incubated with IL-2 only during the standard 5-hour chromium (⁵¹Cr) release assay, while in other experiments effector peripheral blood lymphocytes were preincubated for up to 72 hrs with IL-2 prior to antibody dependent cellular cytotoxicity assay. Target cells were primary uterine serous papillary carcinoma cell lines exposed to Herceptin (concentrations ranging from 1 mg/ml to 5 mg/ml), while controls included the incubation of target cells alone, or with peripheral blood lymphocytes in the presence or absence of IL-2 or MAb, respectively. Rituxan was used as a control monoclonal antibody. Antibody dependent cellular cytotoxicity was calculated as the percentage of killing of target cells observed with monoclonal antibody plus effector peripheral blood lymphocytes, as compared with target cells incubated alone. Each experiment was performed with at least two normal donors, with results from a representative donor presented.

### EXAMPLE 7

Cell Proliferation Assay

Primary uterine serous papillary carcinoma cell lines from patients USPC-1 and USPC-2 were plated at 2,500 cells/well in V-bottomed 96-well plates in the presence or absence of varying concentrations of Herceptin on day 0, using Rituxan as a control. On day 4, cells were pulsed with [H³] thymidine (1 mCi/well) for 6 hrs and then placed in a −20° C. freezer for 1 hr. After thawing at room temperature, cells were harvested using a packard Filtermate Harvester Unifilter-96 and incorporated radioactivity was measured as described (23).

### EXAMPLE 8

HER2/neu Expression by Immunohistology on USPC

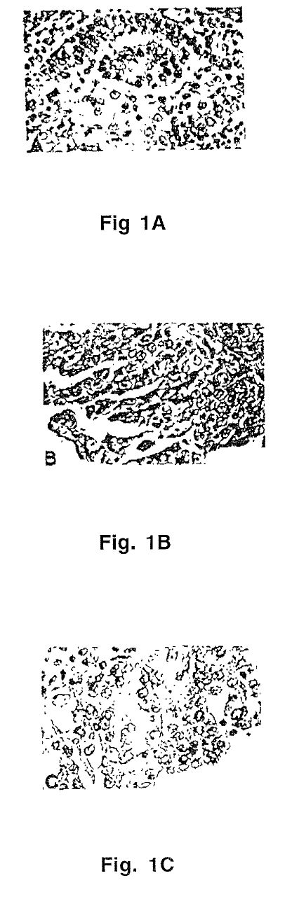

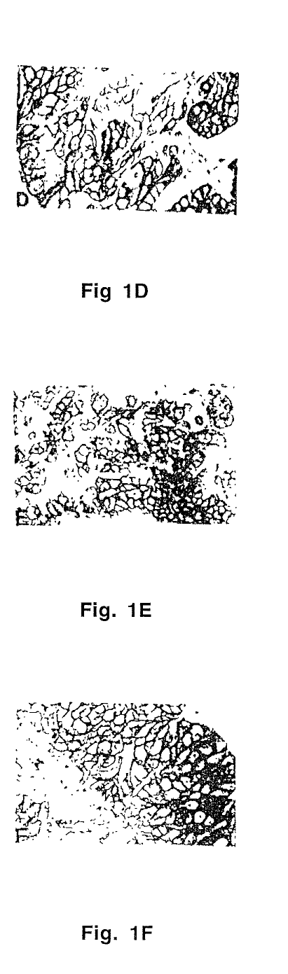

Immunohistochemically detectable HER2/neu protein (i.e., score from 1+ to 3+) was noted in 100% of the uterine serous papillary carcinoma samples evaluated (i.e. 10 out of 10 samples), with 8 out of 10 of the uterine serous papillary carcinoma samples showing moderate (2+, 2 samples) to heavy stain (3+, 6 samples) for HER-2/neu (Table 1). In four cases in which peritoneal metastases were present, HER-2/neu expression was evaluated in both the primary tumor and one metastatic site. In all cases the intensity of staining was the same when the two sites were compared (data not shown). This included one case in which low (1+) HER-2/neu expression was seen and three cases in which high (3+) HER-2/neu expression was seen. All three primary uterine serous papillary carcinoma cell lines established during the study period were from specimens derived from patients harboring uterine serous papillary carcinoma with a score 3+ for HER-2/neu by immunochemistry (i.e., USPC-1, USPC-2 and USPC-3) (FIG. 1). No primary uterine serous papillary carcinoma cell line from patient USPC-4, whose tumor scored 1+ for HER-2/neu by immunohistochemistry was established. The three primary uterine serous papillary carcinoma cell lines were further studied by flow cytometry as well as in biologic assays evaluating the in vitro efficacy of anti-HER-2/neu monoclonal antibody therapy (see below).

### EXAMPLE 9

HER-2/neu Expression by Flow Cytometry on USPC, Serous Papillary Ovarian and Breast Cancer Cell Lines

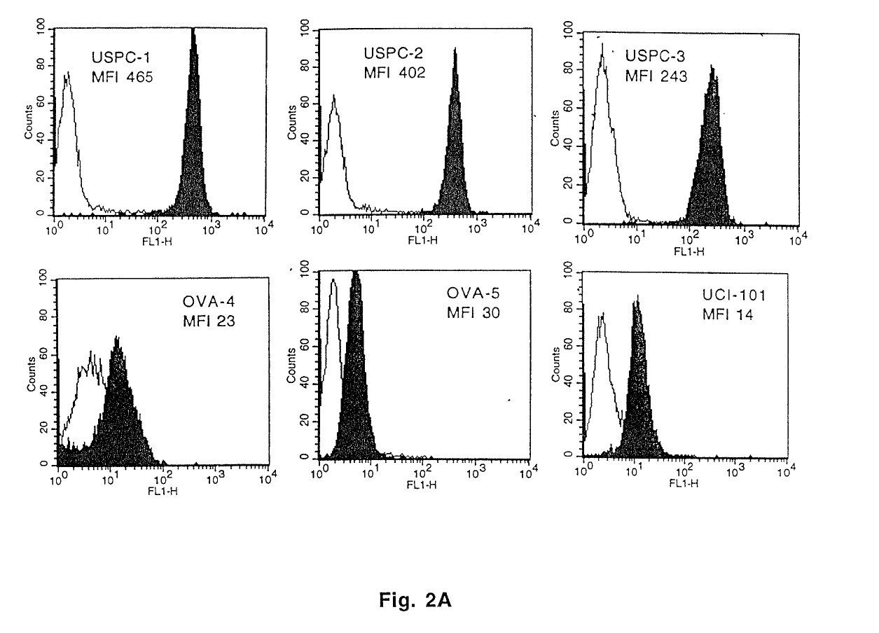

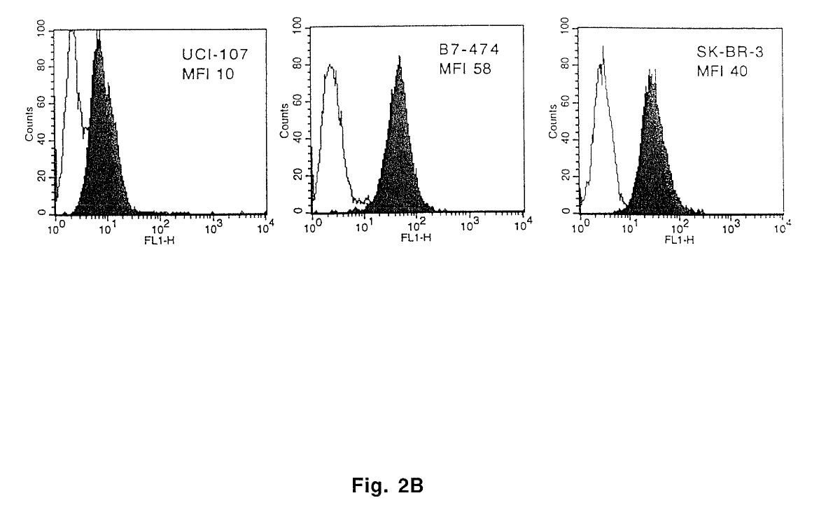

HER-2/neu expression was evaluated by FACS analysis on the primary uterine serous papillary carcinoma cell lines, two primary serous papillary ovarian carcinoma cell lines, and two ovarian (i.e., UCI-101 and UCI-107) and two breast cancer cell lines (i.e., B7-474 and SK-BR3-5), the latter previously being reported to highly over-express HER-2/neu (21,24). In addition, as negative controls, several B cell lines (Epstein-Barr virus-transformed lymphoblastoid cell lines, LCL) established from the same uterine serous papillary carcinoma and ovarian cancer patients from which the tumor cell lines had been established were also studied. Extremely high reactivity against HER-2/neu receptor was found on all three primary uterine serous papillary carcinoma cell lines (100% positive cells for all three USPC), with mean fluorescence intensity (MFI) ranging from to 200 to 450 (USPC-1), 150 to 250(USPC-2) and 170 to 230 (USPC-3), respectively (FIG. 2).

Similarly, all primary and established ovarian cancer and breast cancer cell lines were also found to over-express HER-2/neu by FACS (FIG. 2). Primary and established ovarian and breast cancer cell lines, however, were found to express significantly lower levels of HER-2/neu (average MFI was 10-fold lower) than that expressed by uterine serous papillary carcinoma cells (p<0.001). This finding was particularly remarkable, as the breast cancer cell lines have been previously shown to highly overexpress HER-2/neu and are commonly used as positive controls in several assays evaluating HER-2/neu overexpression (21,24). All autologous B cell lines tested were consistently negative for HER-2/neu expression (data not shown).

### EXAMPLE 10

Uterine Serous Papillary Carcinoma were Resistant to NK Activity but Sensitive to Herceptin-mediated Antibody Dependent Cellular Cytotoxicity

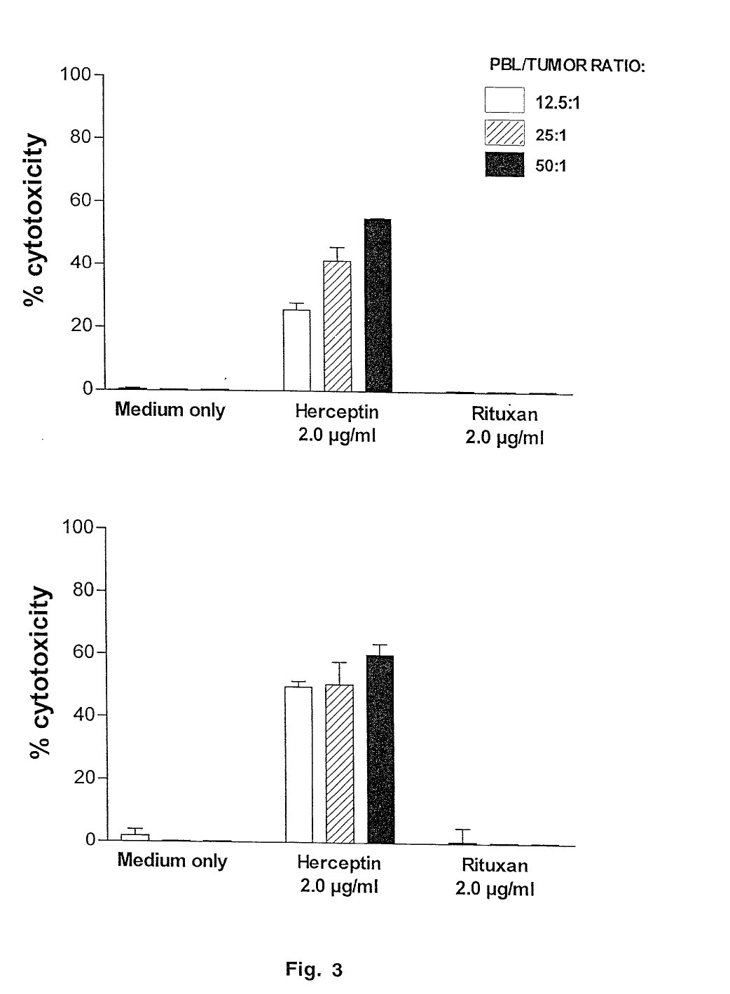

Primary uterine serous papillary carcinoma cell lines were tested for their sensitivity to natural killer cytotoxicity when challenged with peripheral blood lymphocytes collected from several healthy donors in a standard 5 hr ⁵¹Cr release assay. As shown in FIG. 3, uterine serous papillary carcinoma cell lines were consistently found to be resistant to NK-mediated killing when combined with peripheral blood lymphocytes at effector:target (E/T) cell ratios varying from 12.5:1 to 50:1 (range of killing from 0 to 3% with all E/T ratios). Similarly, uterine serous papillary carcinoma cell lines incubated with Rituxan control antibody were not significantly killed (range of killing from 0 to 3% with all E/T ratios)(FIG. 3). In strong contrast, uterine serous papillary carcinoma cell lines were found to be highly sensitive to peripheral blood lymphocytes from heterologous donors combined with Herceptin to mediate antibody dependent cellular cytotoxicity (range of killing from 25% to 60% from 12.5:1 to 50:1 E/T ratio) (FIG. 3). This experiment was repeated five times with similar results.

### EXAMPLE 11

Herceptin-Mediated Antibody Dependent Cellular Cytotoxicity by Autologous Peripheral Blood Lymphocytes

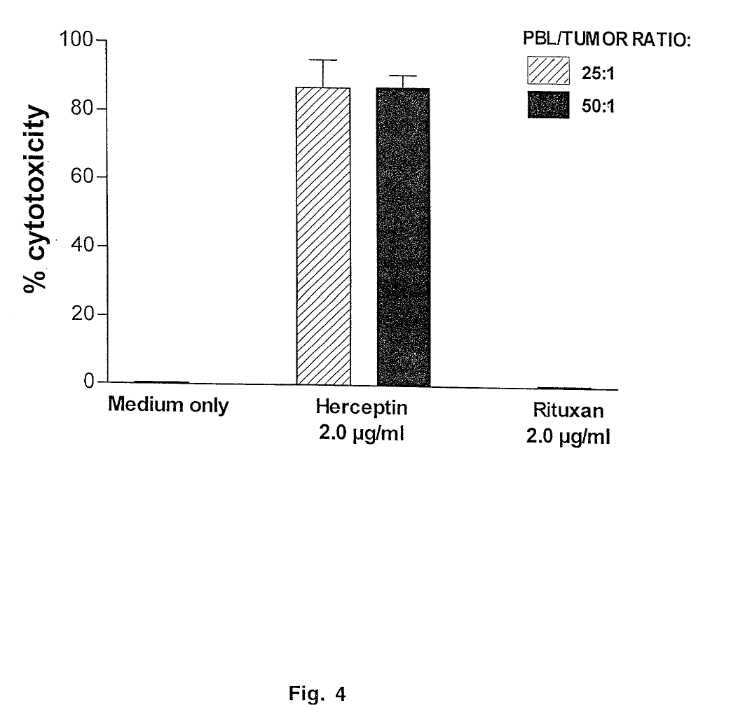

Because in experimental models and human beings (25,26), alteration in number and function of NK cells has been associated with tumor progression, the ability of autologous peripheral blood lymphocytes from patients harboring uterine serous papillary carcinoma to kill tumor cells in the presence or absence of Herceptin was investigated. The USPC-3 cell line was challenged with peripheral blood lymphocytes collected from the patient in a standard 5 hr ⁵¹Cr release assay. Similarly to the results obtained using healthy donor peripheral blood lymphocytes against USPC-1 and USPC-2 cell lines (FIG. 3), USPC-3 was found to be highly resistant to autologous NK-mediated killing at all the effector:target cell ratio tested (i.e., from 25:1 to 50:1, range of killing from 0 to 1% with all E/T ratios), (FIG. 4). The USPC-3 cell line incubated with Rituxan (anti-CD20) control antibody was not significantly killed (range of killing from 0 to 1% with all E/T ratios) (FIG. 4). In contrast, USPC-3 was found to be highly sensitive to Herceptin when combined with autologous peripheral blood lymphocytes to mediate antibody dependent cellular cytotoxicity (range of killing from 35 to 75% from 25:1 to 50:1 E/T ratio)(FIG. 4). This experiment was repeated two times with similar results.

### EXAMPLE 12

Effect of Complement and Physiological Concentrations of IgG on Herceptin-Mediated Antibody Dependent Cellular Cytotoxicity Against USPC

To evaluate primary uterine serous papillary carcinoma cell lines for their sensitivity to complement-mediated cytotoxicity, and to evaluate possible inhibition of antibody dependent cellular cytotoxicity by physiological concentrations of IgG, uterine serous papillary carcinoma cell lines were challenged by adding human serum diluted 1:2 to 1:4 (with or without heat inactivation) in the presence or absence of the effector cells and Herceptin to standard 5 hr ⁵¹Cr release assays

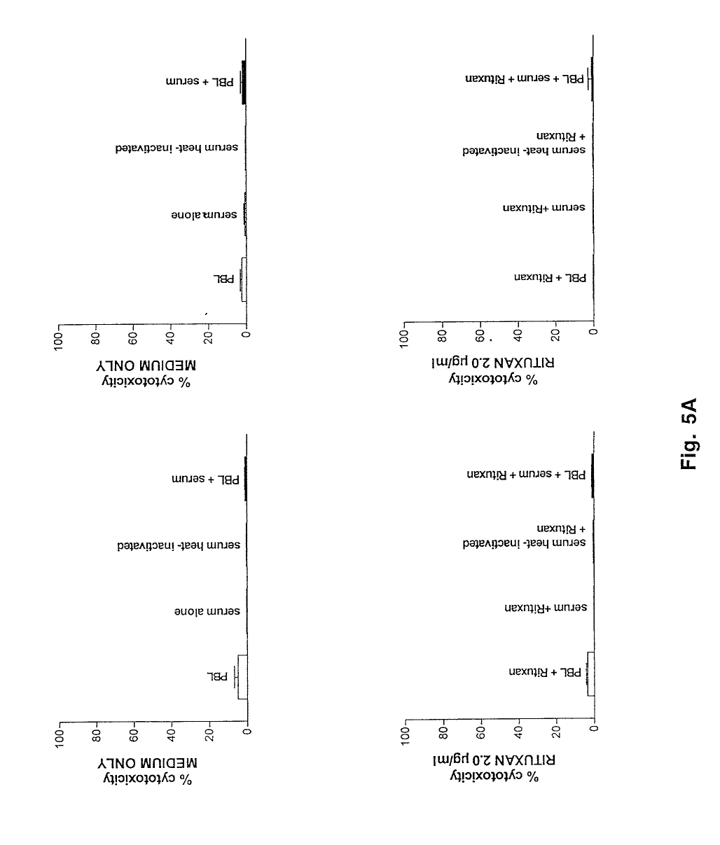

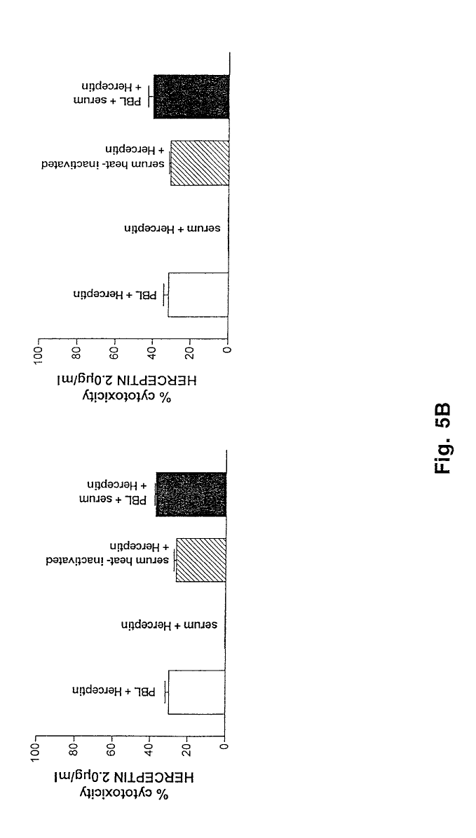

As shown in FIG. 5, addition of untreated serum with or without Herceptin or Rituxan, was not able to induce significant cytotoxicity against USPC-1 (A,C,E) and USPC-2 (B,D,F) cell lines. These data illustrate the lack of significant cytotoxicity mediated by complement proteins in the absence of effector cells. Addition of physiological concentrations of IgG (i.e., heat-inactivated serum diluted 1:2 to 1:4) to peripheral blood lymphocytes in the presence of Herceptin did not significantly alter the degree of antibody dependent cellular cytotoxicity achieved in the presence of Herceptin (FIG. 5). In contrast, addition of untreated serum (diluted 1:2 to 1:4) to peripheral blood lymphocytes in the presence of Herceptin consistently increased Herceptin-mediated cytotoxicity against uterine serous papillary carcinoma (p<0.03)(FIG. 5).

### EXAMPLE 13

IL-2 Enhancement of Antibody Dependent Cellular Cytotoxicity Against USPC

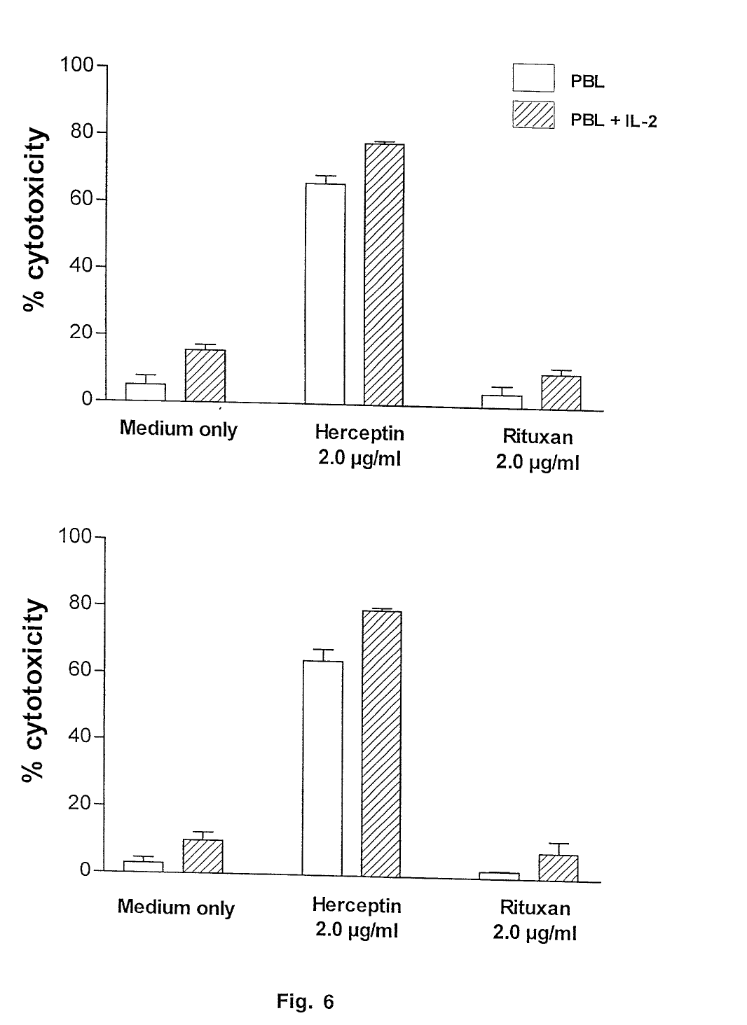

To investigate the effect of low doses of interleukin-2 (IL-2) in combination with Herceptin (2 mg/ml) on1 antibody dependent cellular cytotoxicity against uterine serous papillary carcinoma cell lines, peripheral blood lymphocytes from healthy donors were incubated for 5 hr to 72 hr in the presence of 50 to 100 IU/ml of IL-2. As representatively shown in FIG. 6, Herceptin-mediated antibody dependent cellular cytotoxicity was significantly increased in the presence of low doses of IL-2. Administration of 100 IU/ml of IL-2 to the effector peripheral blood lymphocytes at the start of the assay increased the cytotoxic activity against uterine serous papillary carcinoma cell lines compared to the use of Herceptin alone, while no significant increase in cytotoxicity was detected after 5 hrs' IL-2 treatment in the absence of Herceptin or in the presence of Rituxan control monoclonal antibody (FIG. 6). Longer periods of pre-incubation (72 hr) of effector peripheral blood lymphocytes with IL-2 showed a similar increase in antibody dependent cellular cytotoxicity in the presence of Herceptin. However, a small but significant increase in cytotoxicity was also detectable in the absence of Herceptin and in presence of Rituxan against the uterine serous papillary carcinoma cell line tested, possibly related to lymphokine-activated killer (LAK) activity (FIG. 6).

### EXAMPLE 14

Growth of HER-2/neu Positive Uterine Serous Papillary Carcinoma can be Inhibited by Herceptin in Vitro

Experiments were performed to investigate whether the proliferation of two different HER-2/neu positive uterine serous papillary carcinoma cell lines (USPC-1 and USPC-2) can be inhibited by Herceptin, as compared with Rituxan, which was used as a control. Data presented in Table 2 show this to be the case. The proliferation of both cell lines was significantly inhibited in the presence of Herceptin with the percentage of inhibition varying from 30% to 62% for USPC-1 and from 22% to 52% % for USPC-2 (p>0.05) (Table 2).

TABLE 2
Inhibition of 3Thymidine Uptake by USPC Cells Grown in the
Presence of Herceptin

| Experiment No. | Target Cells | Herceptin Dose | % Inhibition |
|---|---|---|---|
| 1 | USPC-1 | 1 μg/ml | 58% (p < 0.01) |
| | | 2 μg/ml | 61% (p < 0.01) |
| | | 5 μg/ml | 62% (p < 0.01) |
| 2 | USPC-2 | 1 μg/ml | 41% (p < 0.01) |
| | | 5 μg/ml | 52% (p < 0.01) |
| 3 | USPC-1 | 1 μg/ml | 30% (p < 0.03) |
| | | 5 μg/ml | 34% (p < 0.03) |
| 4 | USPC-2 | 1 μg/ml | 22% (p < 0.05) |
| | | 5 μg/ml | 26% (p < 0.05) |

There were 6-8 replicates per group. Rituxan was used as a control monoclonal antibody and gave no inhibition when compared with culture medium alone.

Discussion

In the last few years, several clinical studies have shown that HER-2/neu gene amplification and/or protein overexpression represents the prototype of a stable molecular abnormality endowed with well-characterized functional consequences that is detectable in several of the most common human solid tumors including breast, ovarian, colon, non-small cell lung cancer, prostate and cervical cancer (8, 11 and 19). In the clinical setting, high levels of HER-2/neu in tumor tissue have been associated with shorter patient survival (13-15), resistance to antiestrogens (27) and chemotherapeutic drugs (13-15), and resistance to tumor necrosis factor alpha, activated macrophages and lymphokine activated killer cells (28).

In this study, uterine serous papillary carcinoma, a histologic variant of endometrial cancer characterized by an early intraabdominal and lymphatic spread, an extreme inborn resistance to radiotherapy, chemotherapy and hormonal therapy and a highly aggressive biologic behavior (2-7) commonly overexpresses HER-2/neu. Eight out of 10 (80%) of the uterine serous papillary carcinoma tested in this series by immunohistology on paraffin embedded tissue stained moderately (score 2+) or strongly positive (score 3+) for HER-2/neu surface expression. Patients from whom the primary uterine serous papillary carcinoma cell lines described in this study were established experienced rapid disease progression during chemotherapy (regimen including Taxol, Carboplatin and Doxorubicin) and radiation treatment (data not shown). These findings are therefore consistent with the published evidence regarding the extremely aggressive biologic behavior of this subset of uterine tumors (2-7), and further support the notion that HER-2/neu overexpression may be a major prognostic factor in endometrial cancer (29,30).

All primary uterine serous papillary carcinoma cell lines established and tested in this study were derived from specimens which stained strongly positive for HER-2/neu by immunohistochemistry. In agreement with these results, flow cytometric analysis of all uterine serous papillary carcinoma cell lines demonstrated a striking overexpression of HER-2/neu receptor that was significantly higher than several positive control primary and well-established ovarian cancer cell lines (p<0.001). More surprisingly, however, two breast cancer cell lines (B7-474 and SK-BR-3) known to highly overexpress HER-2/neu and commonly used in many laboratories as positive controls for HER-2/neu receptor expression (21,24), were found to have significantly lower levels of HER-2/neu receptor when compared to primary uterine serous papillary carcinoma cell lines (p<0.001). These data further suggest a correlation between the extremely aggressive biologic behavior of uterine serous papillary carcinoma, their common resistance to standard cytotoxic treatments in vivo and their remarkable overexpression of the HER-2/neu receptor. Nevertheless, this constitutive and striking expression of HER-2/neu in uterine serous papillary carcinoma might turn out to be a useful adjunctive tool to help to differentiate primary uterine serous papillary carcinoma with spread to the ovaries and/or abdominal cavity, as often is the case in the clinic, with the similar but histologically indistinguishable serous papillary ovarian tumors. This differential diagnosis might have important clinical and therapeutic implications.

Primary uterine serous papillary carcinoma studied were found to be highly resistant to killing by natural killer cells and partially resistant to LAK activity (i.e., peripheral blood lymphocytes cultured for up to 72 hrs in 100 IU/ml of IL-2). These data therefore demonstrated that in addition to their high resistance to chemotherapy, radiation treatment and hormonal therapy (2-7), uterine serous papillary carcinoma cells are also intrinsically highly resistant to natural killer activity. Furthermore, complement-mediated tumor cell lysis (in the absence of effector cells) was not observed, which may be due to the presence of membrane-associated complement regulatory proteins such as CD35 (complement receptor 1), CD55 (decay accelerating factor), or CD46 (membrane cofactor protein) on uterine serous papillary carcinoma, as previously reported for other human tumors resistant to complement dependent cytotoxicity (31). In strong contrast, however, all primary uterine serous papillary carcinoma cell lines tested were found to be highly susceptible to antibody dependent cellular cytotoxicity when incubated with heterologous or autologous effector cells in the presence of Herceptin. These data, therefore, demonstrate that although these tumor cells are per se extremely resistant to any standard cytotoxic therapy in the clinic, they remain highly sensitive to the killing activity mediated by NK cells when triggered by HER-2/neu-specific antibody.

In vivo, antibody dependent cellular cytotoxicity applications are known to be dependent upon the availability of the effector cells to interact with the antibody at the target site in the presence of high concentrations of irrelevant human IgG. In this study, antibody dependent cellular cytotoxicity against uterine serous papillary carcinoma was not significantly inhibited by high concentrations (up to 50%) of human serum. In fact, a consistent increase in cytotoxicity was detected in the presence of effector cells and non-heat inactivated human serum. These data, therefore, suggest that in the presence of effector peripheral blood lymphocytes, human serum may augment Herceptin-mediated cytotoxicity against uterine serous papillary carcinoma. Moreover, these results indicate that the binding of Herceptin to the Fc receptor on mononuclear effector cells is of very high affinity and is likely to occur in the in vivo situation.

Treatment of cancer patients with combinations of MAbs and cytokines does not amount to a mere addition to the benefit of each treatment modality alone, but has clearly been demonstrated to have synergistic potential (32,33). Recently, low doses of rIL-2 have been given by continuous infusion or subcutaneously, with remarkable immunologic results coupled with negligible toxicity (34,35). This point is noteworthy because, both in experimental models and in human beings, modulation of both the number and function of NK cells has been previously associated with tumor progression (25,26) and, in addition, substantially suppressed antibody dependent cellular cytotoxicity responses have been reported in several cancer patients (36). Importantly, however, cytotoxicity levels in patients who demonstrate suppressed antibody dependent cellular cytotoxicity can be increased in vitro to levels similar to those of normal donors by prior exposure of effector cells to IL-2 (37). Consistent with this view, a significant increase in antibody dependent cellular cytotoxicity against uterine serous papillary carcinoma was detected after exposure of effector cells from healthy donors as well as one uterine serous papillary carcinoma patient (data not shown) to low doses of IL-2 in vitro for a brief time (i.e., 5 hrs). Longer time periods of incubation (up to 3 days) with IL-2 under the same conditions showed similar results. These data therefore suggest that the administration of low (i.e., non toxic) doses of IL-2 in vivo, giving rise to a lytic effector cell that is markedly enhanced in its function by the addition of an antibody bridge, may significantly increase the efficacy of Herceptin therapy in uterine serous papillary carcinoma patients. Furthermore, on the basis of the high resistance of uterine serous papillary carcinoma to standard cytotoxic anti-cancer therapy, these combined therapies might be particularly important in the treatment of uterine serous papillary carcinoma patients.

Although the majority of previous reports investigating the anti-tumor effects of monoclonal antibodies support the view that efficacy is primarily dependent on immune activation through the Fc receptor (38), others have shown that Herceptin retains about 40% of its anti-tumor activity in FcgRIII^{-/-} mice compared to wild-type mice, indicating that some biological effects of monoclonal antibodies can be independent of Fc receptor binding (39). Consistent with these data, in this study, a significant inhibition in the proliferation of uterine serous papillary carcinoma cell lines was detected by a anti-HER-2/neu monoclonal antibody. These results demonstrated that uterine serous papillary carcinoma behave similarly to ovarian cancer cell lines overexpressing HER-2/neu (16).

In conclusion, the present invention demonstrates that HER-2/neu is highly expressed by uterine serous papillary carcinoma, and that uterine serous papillary carcinoma cells are exquisitely sensitive to Herceptin-mediated antibody dependent cellular cytotoxicity. On the basis of these findings and previous evidence showing a correlation between efficacy of Herceptin therapy in direct proportion to the HER-2/neu overexpression on tumor cells one may postulate that Herceptin is a novel and attractive therapeutic strategy in uterine serous papillary carcinoma patients either for the prevention of recurrence after surgical treatment or for the treatment of metastatic disease. The future design and implementation of clinical trials in this regard will ultimately determine the validity of this approach.

The following references were cited herein.

1. Landis, S. H., Murray, T., Bolden, S., Wingo, P. A. Cancer statistics 1999. CA Cancer J. Clin. 49: 8-31, 1999.

2. Sherman, M. E., Bitterman, P., Rosenshein, N. B., Delgado, G., and Kurman, R. J. Uterine serous carcinoma. A morphologically diverse neoplasm with unifying clinicopathological features. Am. J. Surg. Pathol. 16: 600-610, 1992.

3. Carcangiu, M. L., and Chambers, J. T. Uterine papillary serous carcinoma: a study on 108 cases with emphasis on prognostic significance of associated endometrioid carcinoma, absence of invasion, and concomitant ovarian cancer. Gynecol. Oncol. 47: 298-305, 1992.

4. Goff, B. A., Kato, D., Schmidt, R. A., Ek, M., Ferry, J. A., Muntz, H. G., Cain, J. M., Tamimi, H. K, Figge, D. C., and Greer, B. E. Uterine papillary serous carcinoma: Pattern of metastatic spread. Gynecol. Oncol. 54: 264-268, 1994.

5. Carcangiu, M. L., and Chambers, J. T. Early pathologic stage clear cell carcinoma and uterine papillary serous carcinoma of the endometrium, comparison of clinicopathological features and survival. Int. J. Gynecol. Pathol. 14: 30-38, 1995.

6. Levenback, C., Burke, T. W., Silva, E., Morris, M., Gershenson, D. M. Kavanagh, J. J. and Wharton, J. T. Uterine papillary serous carcinoma (USPC) treated with cisplatin, doxorubicin, and cyclophosphamide (PAC). Gynecol. Oncol. 46:317-321, 1992.

7. Nicklin, J. L., and Copeland, L. J. Endometrial papillary serous carcinoma: pattern of spread and treatment. Clin. Obstet. Gynecol. 39: 686-695, 1996.

8 Slamon, D. J., Godolphin, W., Jones, L. A., Holt, J. A., Wong, S. G., Keith, D. E., Levin, W. J., Stuart, S. G., Udove, J., Ullrich, A., Press, M. F. Studies of the HER-2/neu proto-oncogene in human breast and ovarian cancer. Science.244: 707-712, 1980.

9. Karunagaran, D., Tzahar, E., Beerli, R. R., Chen, X., Graus-Porta, D, Ratzkin B J, Hynes N E, Yarden, Y. ErbB-2 is a common auxiliary subunit of NDF and EGF receptors: implications for breast cencer. EMBO J. 15: 254-264, 1996.

10. Graus-Porta, D., Beerli, R. R., Daly, J. M., Hynes N E. ErbB-2, the preferred heterodimerization partner of all erbB receptors, is a mediator of lateral signaling. EMBO J. 16:1647-1655, 1997.

11. Padhy, L. C., Shih, C., Cowing, D., Finkelstein, R., Weinberg, R. A. Identification of a phosphoprotein specifically induced by the transforming DNA of rat neuroblastomas. Cell. 28: 865-871, 1982.

12. Tal, M., Wetzler, M., Josefberg, Z., Deutch, A., Assaf, D., Kris, R, Shiloh, Y., Givol, D., Schlessinger J. Sporadic amplification of the HER2/neu protooncogene in adenocarcinomas of various tissues. Cancer Res. 48:1517-1520, 1988.

13. Slamon, D. J., Clark, G. M., Wong, S. G., Levin, W. J., Ullrich, A., McGuire, W. L. Human breast cancer: correlation of relapse and survival with amplification of HER-2/neu oncogene. Science. 235: 177-182, 1987.

14. Wright, C., Angus, B., Nicholson, S., Sainsbury, R. C., Cairns, J., Gullick, W. C., Kelly, P., Harris, A. L., Wilson Horne, C. H. Expression of c-erbB-2 oncoprotein: a prognostic indicator in human breast cancer. Cancer Res. 49: 2087-2090, 1989.

15. Berchuck, A., Kamel, A., Whitaker, R., Kerns, B., Olt, G., Kinney, R., Soper, J. T., Dodge, R., Clarke-Pearson, D. L., Marks, P., McKenzie, S., Yin, S., Bast, R. C. Overexpression of HER-2/neu is associated with poor survival in advanced epithelial ovarian cancer. Cancer Res. 50: 4087-4091, 1990.

16. Hellstrom, I., Goodman, G., Pullman, J,. Hellstrom, K E. Overexpression of HER-2 in ovarian carcinomas. Cancer Res. 61: 2420-2423, 2001.

17. Pegram, M. D., Lipton, A., Hayes, D. F., Weber, B. L., Baselga, J. M., Triphaty, D., Baly, D., Baughman, S. A., Twaddell, T., Glaspy, J. A., Slamon, D. J. Phase II study of receptor-enhanced chemosensitivity using recombinant humanized antip185HER2/neu monoclonal antibody plus cisplatin in patients with HER2/neu overexpressing metastatic breast cancer refractory to chemotherapy treatment. J. Clin. Oncol. 16: 2659-2671, 1998.

18. Slamon, D. L., Leyland-Jones, B., Shak, S., Fuchs, H., Paton, V., Pharm, D., Bajamonde, A., Fleming, T., Eiermann, W., Wolter, J., Pegram, M., Baselga, J., Norton, L. Use of chemotherapy plus a monoclonal antibody against HER2 for metastatic breast cancer that overexpresses her2. N. Engl. J. Med. 344: 783-792, 2001.

19. Agus, D. B., Bunn, P. A. Jr, Franklin, W., Garzia, M., Ozols, R. F. HER-2/neu as therapeutic target in non-small cell lung cancer, prostate cancer, and ovarian cancer. Seminars in Oncol. 27: 53-63, 2000.

20. Santin, A. D., Rose, G. S., Hiserodt, J. C., Fruehauf. J., Eck, L. M., Garcia, R. I., Schranz, V., DiSaia, P. J., Pecorelli, S., and Granger, G. A. Effects of tumor necrosis factor-alfa plus interferon-gamma combined with high dose gamma irradiation on the expression of major histocompatibility complex molecules and intercellular adhesion molecule-1 in human ovarian cancers. Int J Cancer 65: 688-694, 1996.

21. Yip, Y. L., Smith, G., Koch, J., Dubel, S., Ward, R. L. Identification of epitope regions recognized by tumor inhibitory and stimulatory anti-Erb-2 monoclonal antibodies: implications for vaccine design. J Immunol. 166: 5271-5278, 2001.

22. Santin, A. D., Hermonat, P. 1., Ravaggi, A., Chiriva-Internati, M., Zhan, D., Pecorelli, S., Parham, G. P., and Cannon, M. Induction of human papillomavirus-specific CD4+ and CD8+ lymphocytes by E7 pulsed autologous dendritic cells in patients with HPV16 and 18 positive cervical cancer. J Virol. 73:5402-5410,1999.

23. Nazaruk, R. A., Rochford, R., Hobbs, M. V. and Cannon, M. J. Functional diversity of the CD8+ T cell response to Epstein-Barr virus (EBV): Implications for the pathogenesis of EBV-associated lymphoproliferative disorders. Blood. 91: 3875-3883, 1998.

24. Mass, R. The role of HER-2 expression in predicting response to therapy in breast cancer. Seminars in Oncol. 27:46-52;2000.

25. Introna, M., Mantovani A. Natural killer cells in human solid tumors. Cancer Metastasis Rev 2: 337-340, 1983.

26. Choe, B., Frost, P., Morrison, N., Rose, N. Natural killer cell activity of prostatic cancer patients. Cancer Invest. 5: 285-288, 1987.

27. Nicholson, S., Wright, C., Sainsbury, J R., Halcrow, P., Kelly, P., Angus, B., Farndon, J R, Harris, A. L. Epidermal growth factor (EGFr) as a marker for poor prognosis in node-negative breast cancer patients: neu and tamoxifen failure. J. Steroid Biochem Mol Biol. 37:811-814, 1990.

28. Ioannides, C. G., Ioannides, M. G., O'Brian, C. A. T-cell recognition of oncogene products: A new strategy for immunotherapy. Mol. Carcinig 6:77-82, 1992.

29. Berchuck, A., Rodriguez, G., Kinney, R. B., Soper, J. T., Dodge, R K, Clarke-Pearson, D. L., Bast, R C. Jr. Overexpression of HER-2/neu in endometrial cancer is associated with advanced stage disease. Am. J. Obstet. gynecol. 164:15-21, 1991.

30. Hetzel, D. J., Wilson, T. O., Keeney, G. L., Roche, P. C., Cha, S. S., Podratz, K. C. HER-2/neu expression: a major prognostic factor in endometrial cancer. Gynecol. Oncol. 47:179-185, 1992.

31. Devine, D. The regulation of complement on cell surfaces. Transfus Med Rev 5:123-131, 1991.

32. Caron, P. C., Lai, L. T., Scheinberg, D. A. Interleukin-2 enhancement of cytotoxicity by humanized monoclonal antibody M195 (anti-CD33) in myelogenous leukemia. Clin. Cancer Res. 1: 63-70, 1995.

33. Hooijberg, E., Sein, J. J., van der Berk, P. C. M., Hart, A. A. M., van der Valk, M. A., Kast, W. M., Melief, C. J. M., Hekman, A. Eradication of large human B cell tumors in nude mice with unconjugated CD20 monoclonal antibodies and Interleukin 2. Cancer Res. 55: 2627-2634, 1995.

34. Stein, R. C., Malkovska, V., Morgan, S., Galaazka, A., Aniszewski, C., Roy, S. E., Shearer, R. J., Marsden, R. A., Bevan, D., Gordon-Smith, E. C., Coombes, R. C. The clinical effects of prolonged treatment of patients with advanced cancer with low-dose subcutaneous interleukin 2. Br. J. Cancer. 63: 275-282, 1991.

35. Caligiuri, M. A., Murray, C., Robertson, M. J., Wang, E., Cochran, K, Cameron, C., Schow, P., Ross, M. E., Klumpp, T. R., Soiffer, R. J., Smith, K. A., Ritz, J. Selective modulation of human natural killer cells in vivo following prolonged infusion of low-doses recombinant interleuikin 2. J. Clin. Invest. 91:123-128, 1993.

36. Ortaldo, J. R., Woodhouse, C. S., Morgan, A. C. Jr., Herberman, R. B., Cheresh, D. A., Reisfeld, R. A. Analysis of effector cells in human antibody-dependent cellular cytotoxicity with murine monoclonal antibodies. J Immunol. 138, 3566-3572, 1987.

37. Honsik, C. J., Jung, G., Reisfeld, R. A. Lymphokine-activated killer cells targeted by monoclonal antibodies to the disialogangliosides GD2 and GD3 specifically lyse humaan tumor cells of neuroectodermal origin. Proc. Natl. Acad. Sci. USA 83:7893-7897, 1986.

38. Clynes, R., Takechi, Y., Moroi, Y., Houghton, A., Ravetch, J. V. Fc receptors are required in passive and active immunity to melanoma. Proc Natl Acad Sci USA 95:652-656, 1998.

39. Clynes, R., Towers, T. L., Persta, L. G., Ravetch, J. V. Fc receptors modulate in vivo cytotoxicity against tumor targets. Nature Med 6:443-446, 2000.

What is claimed is:

1. A method of treating uterine serous papillary carcinoma in an individual in need of such treatment, comprising the step of administering to said individual a therapeutically effective dose of a HER-2/neu antibody.

2. The method of claim 1, wherein said antibody is a monoclonal antibody.

3. The method of claim 2, wherein said antibody is a humanized monoclonal antibody.

4. The method of claim 3, wherein said antibody is Herceptin®.

5. The method of claim 4, wherein said antibody is administered to said individual in a dose of from about 4 mg/kg to about 8 mg/kg.

6. The method of claim 1, further comprising the step of administering a therapeutically effective dose of interleukin-2 to said individual.

7. The method of claim 6, wherein said interleukin-2 is recombinant interleukin-2.

8. The method of claim 6, wherein said dose of interleukin-2 is non-toxic.

9. The method of claim 6, wherein said interleukin-2 is administered to said individual in a dose of from about 1×10⁶ IU/M² to about 10×10⁶ IU/M².

10. A method of differentiating primary uterine serous papillary carcinoma from serous papillary ovarian tumors in an individual, comprising the step of measuring the expression of HER-2/neu in said tissue, wherein the presence of an increased and constitutive expression pattern in said tissue indicates that said tumor is a uterine serous papillary carcinoma.

11. A method of treating uterine serous papillary carcinoma in an individual in need of such treatment, comprising the step of administering to said individual a therapeutically effective dose of a HER-2/neu antibody and a therapeutically effective dose of interleukin-2.

12. The method of claim 11, wherein said antibody is a monoclonal antibody.

13. The method of claim 12, wherein said antibody is a humanized monoclonal antibody.

14. The method of claim 13, wherein said antibody is Herceptin®.

15. The method of claim 14, wherein said antibody is administered to said individual in a dose of from about 4 mg/kg to about 8 mg/kg.

16. The method of claim 11, wherein said interleukin-2 is recombinant interleukin-2.

17. The method of claim 1 1, wherein said dose of interleukin-2 is non-toxic.

18. The method of claim 11, wherein said interleukin-2 is administered to said individual in a dose of from 1×10⁶ IU/M² to about 10×10⁶ IU/M²g.
